(19) United States
(12) Patent Application Publication
(10) Pub. No.: US 2019/0343878 A1
(43) Pub. Date: Nov. 14, 2019

(54) METHOD FOR THE PREPARATION AND PROLONGED STORAGE OF GROWTH FACTORS AND CYTOKINES OBTAINED FROM PLATELET RICH PLASMA

(71) Applicant: Antnor Limited, Toronto (CA)

(72) Inventors: Anthony GALEA, Toronto (CA); Irina BROKHMAN, Vaughan (CA)

(21) Appl. No.: 16/466,763

(22) PCT Filed: Dec. 6, 2017
(86) PCT No.: PCT/CA2017/000262

§ 371 Date: Jun. 5, 2019

(30) Foreign Application Priority Data
Dec. 6, 2016 (CA) 2,950,659

(51) Int. Cl. A61K 35/16; C07K 14/54; C07K 14/495; C07K 14/49; C07K 14/65; A61K 35/19

(52) U.S. Cl. CPC A61K 35/16; C07K 14/5437; C07K 14/5428; A61K 35/19; C07K 14/495; C07K 14/49; C07K 14/65; C07K 14/5403

(57) ABSTRACT

Described is a method of producing a storage stable autologous composition comprising regenerative growth factors and cytokines obtained from platelets. The method comprises the steps of collecting or providing blood from a mammal; delivering the blood to a receptacle; centrifuging the blood to separate a platelet rich plasma component from the blood; collecting the platelet rich plasma component; passing the platelet rich plasma component through a small pore filter to produce the storage stable autologous composition comprising regenerative growth factors and cytokines derived from platelets; and collecting the storage stable autologous composition. The storage stable autologous composition retains regenerative biological activity for up to at least nine months when stored in a frozen state. Also described is a storage stable autologous composition comprising regenerative growth factors and cytokines obtained from platelets produced by the method as described.

### TECHNICAL FIELD

The application is directed generally to medicine, and more particularly to methods for the long term storage of viable compositions useful, in among other things, the treatment of damaged and/or injured connective tissues including chronic tendinosis, chronic muscle tears (tendinitis), cartilage tears, chronic degenerative joint conditions such as osteoarthritis as well as chronic inflammatory skin diseases including, atopic dermatitis and chronic wounds. In addition the viable compositions may be used in dental treatments including dental implants, and for cosmetic applications.

### BACKGROUND

Platelet Rich Plasma (PRP) therapy is employed for the treatment of damaged and/or injured connective tissues, chronic tendinosis, chronic muscle tears and/or chronic degenerative joint conditions and skin inflammatory disorders in a mammal. Platelet Rich Plasma is blood plasma that has been enriched with platelets, typically by centrifugation of blood to isolate a platelet rich component. Platelets contain proteins including growth factors and cytokines that are effective for treating damaged and/or injured connective tissues, chronic tendinosis, chronic muscle tears and/or chronic degenerative joint conditions such as osteoarthritis, and skin inflammatory disorders. These proteins are useful in the treatment of periodontal bone inflammation which has applications for dental implant procedures given the inflammation caused by dental implant hardware. In addition, these proteins are also effective for cosmetic purposes. These cytokines and growth factors include IL-4,10,13, VEGF, PDGF, TGFβ and IGF.

In order for the growth factors and the cytokines to be released from the platelets, the platelets must be activated. PRP fractions are typically collected in the presence of a blood anticoagulant because coagulation of the platelets leads to premature activation of the platelets resulting in the growth factors and the cytokines being released from the platelets prematurely. Some PRP preparation processes take up to thirty minutes. Where no anticoagulant is employed in such processes, the blood may clot prior to the isolation of the PRP component.

For PRP fractions that are collected in the presence of an anticoagulant such as sodium citrate, activation of the platelets can be achieved through a number of means. These means include addition of CaCl, addition of thrombin, freezing and thawing of platelets and ultrasonic waves.

Each of these activation methods has drawbacks. Repeated freeze-thaw cycles lead to degradation of biologically active proteins such as TGFβ and PDGF. Thrombin animal bovine protein can be a source of pathogens or lead to allergic reaction. Human thrombin employed in certain PRP kits is very expensive. Addition of CaCl interferes with the ability to make important measurements of cytokine and growth factor levels and provides a source of impurities into PRP compositions. Lastly, there are regulatory issues with the use of ultrasonic waves and this technique has not been demonstrated to have reliable effectiveness.

There is therefore a need for a more efficient manner of activating platelets thereby releasing the growth factors and cytokines contained therein.

Compositions containing growth factors and cytokines obtained from activated platelets can typically only be stored for about six hours at room temperature before losing biological activity. A typical PRP treatment protocol requires several follow up injections over a period of three to nine months. Typical PRP compositions are not stored between treatments. It is therefore necessary to draw blood from the patient on each visit and often expensive kits are employed for each PRP treatment. This regimen is both invasive and costly.

There is therefore a need for a method of producing a composition comprising growth factors and cytokines obtained from platelets that is effective for treating damaged and/or injured connective tissues, chronic tendinosis, chronic muscle tears and/or chronic degenerative joint conditions such as osteoarthritis, skin inflammatory disorders, cosmetic applications and periodontal bone inflammation, and where the composition is capable of being stored for up to nine months without diminishing the biological activity of the composition.

### SUMMARY OF THE DISCLOSURE

Described is a method for producing a stable composition comprising proteins including cytokines and growth factors obtained from platelet rich plasma. The cytokines and growth factors in the composition retain biological activity and therapeutic effectiveness after prolonged storage for up to at least nine months when frozen. The composition is preferably stored at between −70° C. and −196° C. The composition can be stored at a temperature of −70° C. by employing an ultra-low freezer. Storage at −196° C. can be accomplished by methods known in the art including using liquid nitrogen. After such prolonged storage in a frozen state, the composition remains effective for treating damaged and/or injured connective tissues, chronic tendinosis, chronic muscle tears and/or chronic degenerative joint conditions such as osteoarthritis, skin inflammatory disorders and periodontal bone inflammation. Preferably, the composition comprises IL-4,10,13, VEGF, PDGF, TGFβ. Also described is the composition produced by the method described herein.

According to one aspect, there is provided a method of producing a storage stable autologous composition comprising regenerative growth factors and cytokines obtained from platelets, the method comprising the following steps: collecting blood from a mammal or providing blood from a mammal; delivering the blood to a receptacle preferably including an anticoagulant; centrifuging the blood to separate a platelet rich plasma component from the blood; collecting the platelet rich plasma component; passing the platelet rich plasma component through a small pore filter to produce the storage stable autologous composition comprising regenerative growth factors and cytokines derived from platelets; and collecting the storage stable autologous composition, wherein the storage stable autologous composition retains regenerative biological activity for up to at least nine months when stored in a frozen state.

According to another aspect, there is provided a storage stable autologous composition comprising regenerative growth factors and cytokines obtained from platelets produced by the method described herein.

According to another aspect, there is provided storage stable autologous composition comprising regenerative growth factors and cytokines derived from platelets, the composition comprising biologically active concentrations of IL-4, IL-10, IL-13, PDGF, TGF-β, IGF1 and VEGF, wherein the storage stable autologous composition retains regenerative biological activity for up to at least nine months when stored in a frozen state.

### DESCRIPTION OF THE DRAWINGS

FIG. 1 is a plot of IL-4 concentration in pg/ml showing a comparison of the level of IL-4 in the thrombin and mechanically activated PRP.

FIG. 2 is a plot of IL-10 concentration in pg/ml showing a comparison of the level of IL-10 in the thrombin and mechanically activated PRP.

FIG. 3 is a plot of IL-13 concentration in pg/ml showing a comparison of the level of IL-13 in the thrombin and mechanically activated PRP.

FIG. 4 is a plot of PDGF concentration in pg/ml showing a comparison of the level of PDGF in the thrombin and mechanically activated PRP.

FIG. 5 is a plot of VEGF concentration in pg/ml showing a comparison of the level of VEGF in the thrombin and mechanically activated PRP.

FIG. 6 is a plot of TGβ concentration in pg/ml showing a comparison of the level of TGFβ in the thrombin and mechanically activated PRP.

FIG. 7 is a plot of IL-4 concentration in pg/ml showing a comparison of the level of IL-4 in the fresh and 9 months stored stable solution of Platelet Rich Plasma Ingredients.

FIG. 8 is a plot of IL-10 concentration in pg/ml showing a comparison of the level of IL-10 in the fresh and 9 months stored stable solution of Platelet Rich Plasma Ingredients.

FIG. 9 is a graph showing a comparison between the level of IL-13 in the fresh and 9 months stored stable solution of Platelet Rich Plasma Ingredients.

FIG. 10 is a graph showing a comparison between the level of PGDF in the fresh and 9 months stored stable solution of Platelet Rich Plasma Ingredients.

FIG. 11 is a graph showing a comparison between the level of VEGF in the fresh and 9 months stored stable solution of Platelet Rich Plasma Ingredients.

FIG. 12A is a graph showing a comparison between the level of TGFβ in the fresh and 9 months stored stable solution of Platelet Rich Plasma Ingredients.

FIG. 12B is a graph indicating instability of TGF-β1 concentration upon different conditions.

FIG. 13 is a graph showing a comparison between the level of IGF1 in the fresh and 9 months stored stable solution of Platelet Rich Plasma Ingredients.

### DETAILED DESCRIPTION OF THE INVENTION

The disclosure relates to a method for producing a stable composition comprising a platelet rich plasma component wherein the composition retains biological activity and therapeutic efficacy after prolonged storage for up to at least nine months when stored preferably at between −70° C. and −196° C. for treating damaged and/or injured connective tissues, chronic tendinosis, chronic muscle tears and/or chronic degenerative joint conditions such as osteoarthritis, skin inflammatory disorders and periodontal bone inflammation caused by for example dental implants.

The platelet rich plasma component of the composition preferably includes the following therapeutically active proteins: IL-4⁵, IL-10^{6,7}, IL-13⁸, PDGF⁹, TGF-β^{10,11}, IGF1 and VEGF.^{12,13,14}

IL-4,10,13, PDGF, IGF1, TGF-β are contained in the platelet a-granules and are delivered to the composition by the platelet rich plasma component. The ability to store the composition for a prolonged period of time provides a potent and accessible bioactive autologous product that is efficient and cost effective to administer to patients. Thus, the storage stable composition of therapeutically active proteins included in the platelet rich plasma component provides a source of regenerative biological factors and anti-inflammatory cytokines and growth factors. The composition also provides a powerful and cost effective treatment of degenerative conditions including osteoarthritis, chronic tendinosis and chronic muscle tears as well as skin inflammatory disorders and periodontal bone inflammation caused by dental procedures such as dental implants given the inflammation caused by dental implant hardware in such procedures. In addition, the composition also provides powerful and cost effective cosmetic applications.

As used herein, “treatment” includes regenerative and palliative treatment, wherein pain and/or inflammation is reduced in the subject.

The described method for producing and storing the stable composition of platelet rich plasma components preferably including regenerative growth factors and cytokines derived from platelets comprises the step of collecting a mammal's autologous physiological fluid, preferably blood by an aseptic technique. Preferably, the mammal is a human. However the compositions and methods hereof are also suitable for a wide range of veterinary applications, for example for the treatment of horses, dogs and camels.

The site of venipuncture and the surface of the collection tubes may be cleaned with a 2 percent tincture of iodine solution. Before any cleansing of the site is begun, the patient may be asked about any allergy to iodine. The collection container covers are cleaned with 80% alcohol solution also to avoid possible contamination before blood collection.

The collection of blood is preferably carried out in the presence of an anticoagulant. Preferably, the anticoagulant is 4% citric acid. Preferably, the ratio is 9.5 parts of whole blood (9.5 cc) : 0.5 (0.5 cc) of 4% citric acid. The blood is then subjected to centrifugation preferably for about 30 sec, at about 7500 rpm to isolate the PRP fraction. However, a person skilled the art will appreciate that the blood can be processed or the PRP fraction can be isolated using commercially available kits such as Harvest™ and Arthrex™.

Preferably, the isolated PRP fraction is harvested and activated mechanically by passing the PRP fraction through a small pore size filter and collected in a sterile plastic vial. Preferably the pore size of the filter is in the range of about 0.15 μm to about 1 μm. Most preferably the pore size of the filter is about 0.22 μm. An example of a filter that is suitable for carrying out this method is Millipore Corporation, Millex-GP 33 mm PES 0.22 um Sterile, Catalog N SLGPM33RS. The isolated PRP fraction is preferably passed through the filter by adding the PRP fraction to a syringe and delivering the PRP fraction through the filter through the action of the syringe.

Passing the platelet rich plasma component through the small pore filter mechanically activates the platelets in the PRP fraction. This releases the cytokines and growth factors from the platelets. Platelet debris is caught in the filter. The liquid composition that is obtained after the PRP fraction passes through the filter is the storage stable autologous composition comprising regenerative growth factors and cytokines derived from platelets.

Preferably, the storage stable autologous composition is then passed a second time through a separate small pore filter having a pore size in the range of about 0.15 m to about 1 μm, and preferably about 0.22 m. It is possible to employ the same filter as for the first pass through of the PRP fraction. However, this is not preferred due to potential filter occlusion.

The next step involves collecting the storage stable autologous composition and dividing it into aliquots for future processing using a sterile technique or for storage. The procedure is carried out in a sterile environment (laminar flow hood with HEPA filters). Two to four cc of the storage stable autologous composition containing biologically active agents are carefully drawn by sterile syringe and needle. Prolonged storage of PRP ingredients containing product is accomplished by freezing the aliquots. Preferably the aliquots are frozen at a temperature in the range of about −70° C. to about −196° C. and most preferably at about −80° C. followed by storing for up to 9 months while maintaining acceptable biological activity and therapeutic efficacy of the storage stable autologous composition. The storage stable autologous composition can be frozen and stored while maintaining acceptable biological activity and therapeutic efficacy for at least nine months.

The method of PRP mechanical activation of the present disclosure leads to the same cytokines and growth factors in the compositions as a thrombin activation.

This composition can be stored in a frozen state for at least nine months without decreasing its biological activity and reducing IL-4, IL-10, IL-13, PDGF, TGF-β, IGF1 and VEGF concentrations.

The properties of the storage stable autologous composition are further described with the aid of the following illustrative examples.

### EXAMPLE 1—COMPARISON OF LEVELS OF CYTOKINES AND GROWTH FACTORS OBTAINED THROUGH MECHANICAL ACTIVATION OF PLATELETS TO LEVELS OF CYTOKINES AND GROWTH FACTORS OBTAINED THROUGH ACTIVATION OF PLATELETS WITH BOVINE THROMBIN

Blood was obtained from patients and a PRP fraction of the blood was obtained from each patient according to the method of the present disclosure.

For each patient, the platelets in the PRP fraction were activated according to the following procedure: Bovine thrombin was added: 100 units for 1 cc PRP. Samples were incubated at room temperature for 10 min to allow plasma to clot.

For each patient, the platelets in the PRP fraction were activated mechanically according to the procedure set out in the present disclosure by passing the PRP fraction through a filter having a pore size of about 0.22 μm. The PRP fraction was passed through a 0.22 μm filter a second time.

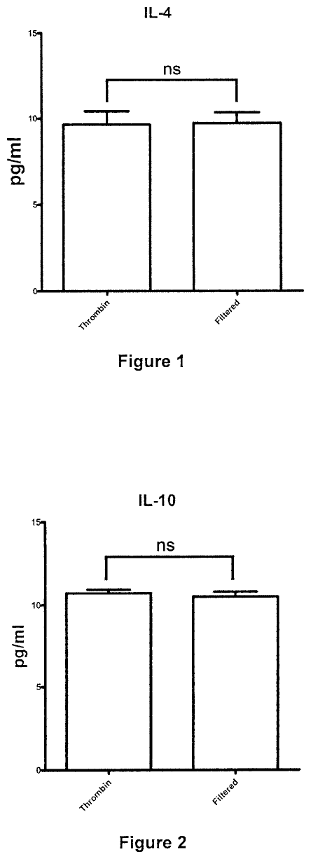

The data obtained is summarized in FIGS. 1-6.

FIG. 1 is a plot of IL-4 concentration in pg/ml showing a comparison of the level of IL-4 in the composition obtained from platelets activated by thrombin and platelets activated from the PRP fraction. The results show that there is no statistically significant difference in the level of IL-4 in the composition where the platelets were activated by thrombin compared to the level of IL-4 in the composition where the platelets were activated mechanically according to the method of the present disclosure.

FIG. 2 is a plot of IL-10 concentration in pg/ml showing a comparison of the level of IL-10 in the composition obtained from platelets activated by thrombin and platelets activated from the PRP fraction. The results show that there is no statistically significant difference in the level of IL-10 in the composition where the platelets were activated by thrombin compared to the level of IL-10 in the composition where the platelets were activated mechanically according to the method of the present disclosure.

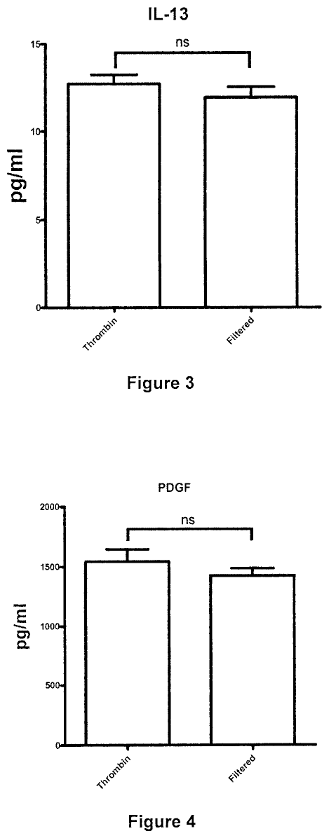

FIG. 3 is a plot of IL-13 concentration in pg/ml showing a comparison of the level of IL-13 in the composition obtained from platelets activated by thrombin and platelets activated from the PRP fraction. The results show that there is no statistically significant difference in the level of IL-13 in the composition where the platelets were activated by thrombin compared to the level of IL-13 in the composition where the platelets were activated mechanically according to the method of the present disclosure.

FIG. 4 is a plot of PDGF concentration in pg/ml showing a comparison of the level of PDGF in the composition obtained from platelets activated by thrombin and platelets activated from the PRP fraction. The results show that there is no statistically significant difference in the level of PDGF in the composition where the platelets were activated by thrombin compared to the level of PDGF in the composition where the platelets were activated mechanically according to the method of the present disclosure.

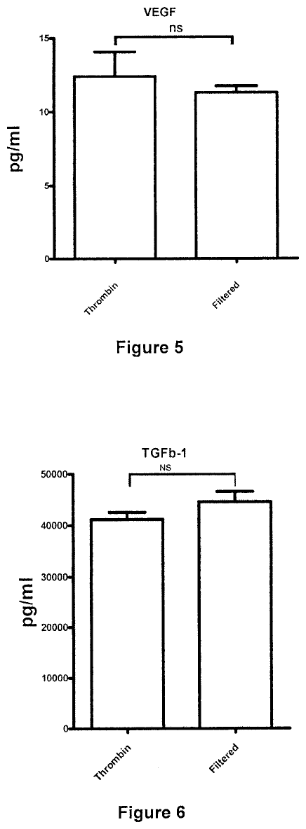

FIG. 5 is a plot of VEGF concentration in pg/ml showing a comparison of the level of VEGF in the composition obtained from platelets activated by thrombin and platelets activated from the VEGF fraction. The results show that there is no statistically significant difference in the level of VEGF in the composition where the platelets were activated by thrombin compared to the level of VEGF in the composition where the platelets were activated mechanically according to the method of the present disclosure.

FIG. 6 is a plot of TGFβ concentration in pg/ml showing a comparison of the level of TGF in the composition obtained from platelets activated by thrombin and platelets activated from the TGβ fraction. The results show that there is no statistically significant difference in the level of TGFβ in the composition where the platelets were activated by thrombin compared to the level of TGFβ in the composition where the platelets were activated mechanically according to the method of the present disclosure.

### EXAMPLE 2:—COMPARISON OF LEVELS OF CYTOKINES AND GROWTH FACTORS OBTAINED THROUGH MECHANICAL ACTIVATION OF PLATELETS IN FRESH AUTOLOGOUS COMPOSITION AND IN COMPOSITION STORED FOR 9 MONTHS AT −80° C.

The storage stable autologous composition was obtained according to the method of the present disclosure. The composition was divided into aliquots of 2-4 cc by carefully drawing of the composition by a sterile syringe and needle. The aliquots were stored at −80° C. for 9 months.

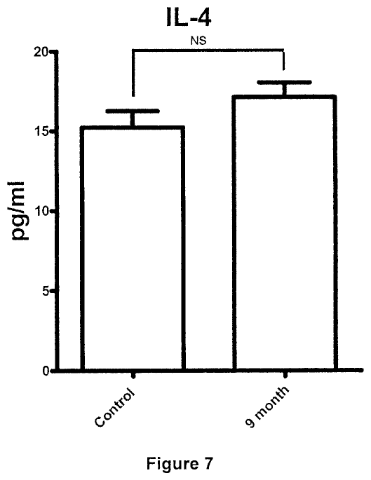

FIG. 7 is a plot of IL-4 concentration in pg/ml showing a comparison of the level of IL-4 in the fresh storage stable autologous composition to the level of IL-4 in the storage stable autologous composition stored for nine months at −80° C. The results show that there is no statistically significant difference in the respective levels and that accordingly the concentration of IL-4 in the storage stable autologous composition is preserved after storage for nine months at −80° C.

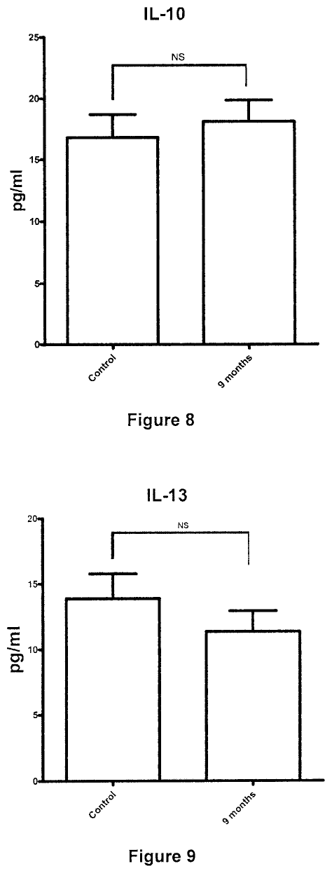

FIG. 8 is a plot of IL-10 concentration in pg/ml showing a comparison of the level of IL-10 in the fresh storage stable autologous composition to the level of IL-10 in the storage stable autologous composition stored for nine months at −80° C. The results show that there is no statistically significant difference in the respective levels and that accordingly the concentration of IL-10 in the storage stable autologous composition is preserved after storage for nine months at −80° C.

FIG. 9 is a plot of IL-13 concentration in pg/ml showing a comparison of the level of IL-13 in the fresh storage stable autologous composition to the level of IL-13 in the storage stable autologous composition stored for nine months at −80° C. The results show that there is no statistically significant difference in the respective levels and that accordingly the concentration of IL-13 in the storage stable autologous composition is preserved after storage for nine months at −80° C.

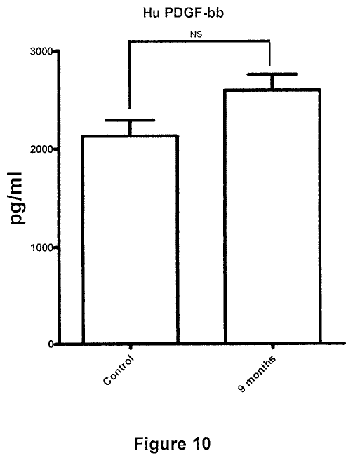

FIG. 10 is a plot of PGDF concentration in pg/ml showing a comparison of the level of PGDF in the fresh storage stable autologous composition to the level of PGDF in the storage stable autologous composition stored for nine months at −80° C. The results show that there is no statistically significant difference in the respective levels and that accordingly the concentration of PGDF in the storage stable autologous composition is preserved after storage for nine months at −80° C.

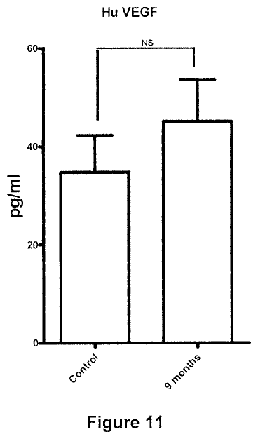

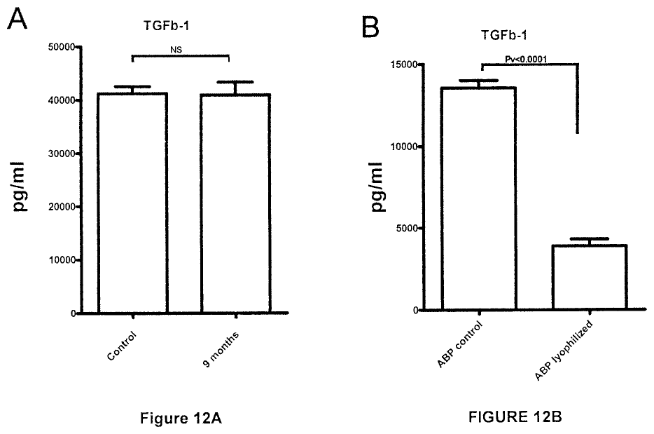

FIG. 11 is a plot of VEGF concentration in pg/ml showing a comparison of the level of VEGF in the fresh storage stable autologous composition to the level of VEGF in the storage stable autologous composition stored for nine months at −80° C. The results show that there is no statistically significant difference in the respective levels and that accordingly the concentration of VEGF in the storage stable autologous composition is preserved after storage for nine months at −80° C.

FIG. 12 A is a plot of TGβ-1 concentration in pg/ml showing a comparison of the level of TGFβ in the fresh storage stable autologous composition to the level of TGFβ-1 in the storage stable autologous composition stored for nine months at −80° C. The results show that there is no statistically significant difference in the respective levels and that accordingly the concentration of TGFβ-1 in the storage stable autologous composition is preserved after storage for nine months at −80° C.

FIG. 12 B is a plot of TGFβ-1 concentration in pg/ml showing a comparison of the level of TGFβ in the fresh and lyophilized autologous blood product (a combination of incubated blood and PRP). The results show that a lyophilizing method leads to significant decreasing of the level of TGFβ-1 whereas the freezing method (A) preserves normal concentration.

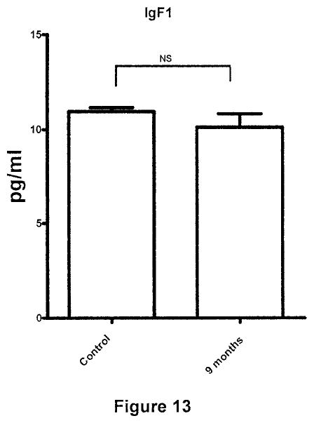

FIG. 13 is a plot of IGF1 concentration in pg/ml showing a comparison of the level of IGF1 in the fresh storage stable autologous composition to the level of IGF1 in the storage stable autologous composition stored for nine months at −80° C. The results show that there is no statistically significant difference in the respective levels and that accordingly the concentration of IGF1 in the storage stable autologous composition is preserved after storage for nine months at −80° C.

Although the invention has been described with reference to illustrative embodiments, it is to be understood that the invention is not limited to these precise embodiments. Numerous modifications, variations, and adaptations may be made to the particular embodiments of the invention described above without departing from the scope of the invention. The scope of the claims should not be limited by the preferred embodiments set forth in the examples, but should be given the broadest interpretation consistent with the description as a whole.

### REFERENCES

1. Fernandes J C, Martel-Pelletier J, Pelletier J P. The role of cytokines in osteoarthritis pathophysiology. Biorheology. 2002; 39(1-2)237-46.
2. Fredberg U, Stengaard-Pedersen K. Chronic tendinopathy tissue pathology, pain mechanisms, and etiology with a special focus on inflammation. Scand J Med Sci Sports. 2008 February; 18(1):3-15.
3. Loell I, Lundberg I E. Can muscle regeneration fail in chronic inflammation: a weakness in inflammatory myopathies? J Intern Med. 2011 March; 269(3):243-57.
4. Rita E, Mirza, Milie M. Fang,1 William J, Timothy J. Koh. Blocking Interleukin-1β Induces a Healing-Associated Wound Macrophage Phenotype and Improves Healing in Type 2 Diabetes. Diabetes. 2013 July; 62(7): 2579-2587.
5. Eming S A, Krieg T, Davidson J M. Inflammation in wound repair: molecular and cellular mechanisms. J Invest Dermatol. 2007; 127:514.
6. Dinarello C A. Interleukin-1, interleukin-1 receptors and interleukin-1 receptor antagonist. Int Rev Immunol. 1998; 16(5-6):457-99.
7. Mosmann T R, Cherwinski H, Bond M W, Giedlin M A, Coffman R L. Two types of murine helper T cell clone. I. Definition according to profiles of lymphokine activities and secreted proteins. J Immunol. 1986 Apr. 1; 136(7):2348-57.
8. Brandtzaeg P1, Osnes L, Ovstebø R, Joø G B, Westvik A B, Kierulf P. Net inflammatory capacity of human septic shock plasma evaluated by a monocyte-based target cell assay: identification of interleukin-10 as a major functional deactivator of human monocytes. J Exp Med. 1996 Jul. 1; 184(1):51-60.
9. Clarke C J1, Hales A, Hunt A, Foxwell B M. IL-10-mediated suppression of TNF-alpha production is independent of its ability to inhibit NF kappa B activity. Eur J Immunol. 1998 May; 28(5):1719-26.
10. de Waal Malefyt R, Figdor C G, Huijbens R, Mohan-Peterson S, Bennett B, Culpepper J, Dang W, Zurawski G, de Vries J E. Effects of IL-13 on phenotype, cytokine production, and cytotoxic function of human monocytes. Comparison with IL-4 and modulation by IFN-gamma or IL-10. J Immunol. 1993 Dec. 1; 151(11):6370-81.
11. Alvarez R H1, Kantarjian H M, Cortes J E. Biology of platelet-derived growth factor and its involvement in disease. Mayo Clin Proc. 2006 September; 81(9):1241-57.
12. Roberts A B, Flanders K C, Kondaiah P, Thompson N L, Van Obberghen-Schilling E, Wakefield L, Rossi P, De Crom-Brugghe B, Heine U I, Sporn M B: Transforming growth factor beta: biochemistry and roles in embryogenesis, tissue repair and remodeling, and carcinogenesis. Rec Prog Horm Res 44:157-197, 1988
13. Roberts A B, Heine U I, Flanders K C, Sporn M B: TGF-beta: Major role in regulation of extracellular matrix. Ann NY Acad Sci. “Structure, Molecular Biology and Pathology of Collagen”. 580:225-232, 1990.
14. Roffi, A., Filardo, G., Assirelli, A., Cavallo, C., Cenacchi, A., Facchini, A., Grigolo, B., Kon, E. and Marcacci, M. Does Platelet-Rich Plasma Freeze-Thawing Influence Growth Factor Release and Their Effects on Chondrocytes and Synoviocytes? BioMed Research International Volume 2014.15. Gospodarowicz, D., Abraham, J. A., and Schilling, J. Isolation and characterization of a vascular endothelial cell mitogen produced by pituitary-derived folliculo stellate cells. Proc. Natl. Acad. Sci. USA 86, 7311-7315, 1989.
14. Fufa D, Shealy B, Jacobson M, et al. Activation of platelet-rich plasma using soluble Type I collagen. J Oral Maxillofac Surg 2008; 66:684-90
15. Marinello F. Serum or plasma samples? The “Cinderella” role of blood collection procedures: preanalytical methodological issues influence the release and activity of circulating matrix metalloproteinases and their tissue inhibitors, hampering diagnostic trueness and leading to misinterpretation. Arterioscler Thromb Vasc Biol. 2008 April; 28(4):611-4.

The content of these references is hereby incorporated by reference.

1. A method of producing a storage stable autologous composition comprising regenerative growth factors and cytokines obtained from platelets, the method comprising the following steps:
providing blood from a mammal;
delivering the blood to a receptacle;
centrifuging the blood to separate a platelet rich plasma component from the blood;
collecting the platelet rich plasma component;
passing the platelet rich plasma component through a small pore filter to produce the storage stable autologous composition comprising regenerative growth factors and cytokines derived from platelets; and
collecting the storage stable autologous composition,
wherein the storage stable autologous composition retains regenerative biological activity for up to at least nine months when stored in a frozen state.

2. The method of claim 1 wherein the storage stable autologous composition is stored at a temperature in the range of −70° C. to −196° C.

3. The method of claim 1 wherein the storage stable autologous composition is stored at −80° C.

4. The method of claim 1 wherein the storage stable autologous composition is stored at −196° C.

5. The method of claim 1 wherein the storage stable autologous composition is delivered a second time through a second small pore filter having a pore size in the range of 0.15 μm to 1 μm.

6. The method of claim 1 wherein the pore size of the small pore filter is in the range of 0.15 μm to 1 μm.

7. The method of claim 1 wherein the pore size of the small pore filter is about 0.22 μm.

8. The method of claim 1 wherein the growth factors and cytokines include IL-4, IL-10, IL-13, PDGF, TGF-β, IGF1 and VEGF.

9. The method according to claim 1 wherein the receptacle includes a quantity of an anticoagulant.

10. The method according to claim 9 wherein the anticoagulant is about 4% citric acid.

11. The method according to claim 1 wherein the receptacle is a tube.

12. The method of claim 1 further including the step of dividing the storage stable autologous composition into aliquots prior to freezing and storing the storage stable autologous composition.

13. The method of claim 1 wherein the aliquots are about 10 cc in volume of the storage stable autologous composition.

14. The method according to claim 1, wherein the step of centrifuging the blood to separate a platelet rich plasma component from the blood is carried out for about thirty seconds at about 7500 rpm.

15. A storage stable autologous composition comprising regenerative growth factors and cytokines derived from platelets produced by the method of claim 1.

16. A storage stable autologous composition comprising regenerative growth factors and cytokines derived from platelets produced by the method of claim 9.

17. A storage stable autologous composition comprising regenerative growth factors and cytokines derived from platelets, the composition comprising biologically active concentrations of IL-4, IL-10, IL-13, PDGF, TGF-β, IGF1 and VEGF, wherein the storage stable autologous composition retains regenerative biological activity for up to at least nine months when stored in a frozen state at a temperature in the range of −70° C. to −196° C.

18. A storage stable autologous composition according to claim 17 wherein the storage temperature is −80° C.
